(19) United States
(12) Patent Application Publication
(10) Pub. No.: US 2024/0215865 A1
(43) Pub. Date: Jul. 4, 2024

(54) DETERMINING THE QUALITY OF SETTING UP A HEADSET FOR CRANIAL ACCELEROMETRY

(71) Applicants: Brainlab AG, Munich (DE); Jan Medical, Inc., Mountain View, CA (US)

(72) Inventors: Nicolas MÜLLER, Munich (DE); Stefan HAGER, Munich (DE); Stephan MITTERMEYER, Munich (DE); Dave M. COOK, Mountain View, CA (US); Ray SCHUMACHER, Mountain View, CA (US)

(21) Appl. No.: 18/686,497

(22) PCT Filed: Oct. 14, 2021
(86) PCT No.: PCT/EP2021/078408

§ 371 Date: Feb. 26, 2024

(51) Int. Cl. A61B 5/11; A61B 5/00; A61B 5/0205; G16H 40/67; A61B 5/024

(52) U.S. Cl. CPC A61B 5/1114; A61B 5/0205; A61B 5/1126; A61B 5/6803; A61B 5/6844; A61B 5/7221; G16H 40/67; A61B 5/024; A61B 2562/0219; A61B 2562/0247; A61B 2562/046; A61B 2562/066

(57) ABSTRACT

Disclosed is a computer-implemented method of analyzing the setup of an array of acceleration sensors on an anatomical body part of a patient encompasses acquisition of acceleration data using a cranial headset having acceleration sensors and determining an appropriate contact of the acceleration sensor to the patient's head, detecting, marking, filtering out, or removing different kinds of noise as well as acceleration signals which are considered to be due to gross patient movement. This is done to determine whether the setup of the headset on the patient's head is good enough to generate measurement signals of acceptable quality. If this is the case, the headset is subsequently used to acquire continuous datasets of acceleration data which is then evaluated to determine the patient's physiological status.

### FIELD OF THE INVENTION

The present invention relates to a computer-implemented method of analyzing the setup of an array of acceleration sensors on an anatomical body part of a patient, a corresponding computer program, a computer-readable storage medium storing such a program and a computer executing the program, as well as a medical system comprising an electronic data storage device and the aforementioned computer.

### TECHNICAL BACKGROUND

Determination of brain anomalies such as a large vessel occlusion, aneurysm, or vasospasm is typically done using imaging modalities (e.g., CT, MRI, etc.) and other technical approaches such as EEG, Doppler sonography, cerebral ultrasound, or auscultation via microphones. Most of these approaches can only be performed in a clinical environment and thus there is a need of a mobile application for enabling an aid to diagnosis directly at the patient's site. Cranial accelerometry has already been described as a potential solution but comes with a sensitivity to interferences. With the proposed methods, this is addressed providing a user-friendly and reliable system to generate data suitable for analysis.

Acceleration sensors are highly sensitive and thus prone to artefacts. For a meaningful conclusion drawn from the cranial acceleration recording, high quality and mostly artefact-free data is required for analysis.

The present invention has the object of providing a method of determining the quality of setting up a system for cranial accelerometry, for example such that the amount of artefacts in signals acquired using the system is reduced as far as possible.

The presented invention can be used for enhanced setup of a cranial accelerometry headset in triaging procedures of patients directly on location to prepare for execution of established triage methods such as CPSS (Cincinnati Prehospital Stroke Scale), RACE (Rapid Arterial oCclusion Evaluation), or LAMS (Los Angeles Motor Scale).

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given which shall not be understood to limit the invention only to the features or a combination of the features described in this section.

The disclosed computer-implemented method of analyzing the setup of an array of acceleration sensors on an anatomical body part of a patient encompasses acquisition of acceleration data using a cranial headset having acceleration sensors and determining an appropriate contact of the acceleration sensor to the patient's head, detecting, marking, filtering out, or removing different kinds of noise as well as acceleration signals which are considered to be due to gross patient movement. This is done to determine whether the setup of the headset on the patient's head is good enough to generate measurement signals of acceptable quality. If this is the case, the headset is subsequently used to acquire continuous datasets of acceleration data which is then evaluated to determine the patient's physiological status.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method of analyzing the setup of an array of acceleration sensors on an anatomical body part of a patient. The anatomical body part in an example is the patient's head but may be any other anatomical body part. The method according to the first aspect comprises for example executing, on at least one processor of at least one computer (for example at least one computer being part of a portable system, for example a handheld device such as a mobile phone or tablet computer), the following exemplary steps which are executed by the at least one processor.

In a (for example first) exemplary step, acceleration measurement data is acquired which describes acceleration values acquired using the acceleration sensors.

In a (for example second) exemplary step, sensor contact data is acquired which describes a quality of a contact between at least one of the acceleration sensors comprised in the array of acceleration sensors on the one hand and the anatomical body part on the other hand. For example, the sensor contact data is acquired based on the acceleration measurement data, for example by acceleration sensor combinations, correlation of ambient sound on the acceleration measurement data or by principal component analysis or independent component analysis to determine signal and noise components on the acceleration measurement data, or by skin resistance measurement.

In an example of the method according to the first aspect, determining the sensor contact data comprises the following steps:

Contact pressure data is acquired which describes pressure values detected by each of pressure sensors comprised in an array of pressure sensors after positioning each of the pressure sensors on the anatomical body part. For example, the array of pressure sensors and the array of acceleration sensors are comprised in the same device and for example have a predetermined, for example at least one of fixed or known, spatial relationship to each other.

Pressure threshold data is acquired which describes at least one threshold value of the pressure values detected by each of the pressure sensors. The at least one threshold value serves as an indication of acceptable pressure values.

Pressure change data is determined based on the contact pressure data, wherein the pressure change data describes a first-order temporal derivative of the pressure values detected by each of the pressure sensors.

Pressure change threshold data is acquired which describes at least one threshold value of the first-order temporal derivative of the contact pressure data. The sensor contact data is then determined for example based on the contact pressure data and the pressure threshold data and the pressure change data and the pressure change threshold data. For example, a predetermined (for example desired, specifically sufficient or good) quality of the contact is determined when the pressure values detected by each of the pressure sensors is larger than the at least one threshold value of the pressure values and if the first-order temporal derivative of the pressure values is smaller than the at least one threshold value of the first-order temporal derivative of the contact pressure data.

In an example of the method according to the first aspect, pressure quality data is determined based on the contact pressure data and the pressure threshold data by comparing the pressure values detected by each of the pressure sensors to the at least one threshold value of the pressure values, wherein the pressure quality data is determined to indicate a predetermined quality of the pressure values detected by each of the pressure sensors if the comparison results in that the pressure values detected by each of the pressure sensors have predetermined relationship to the at least one threshold value of the pressure values.

In an example of the method according to the first aspect, background noise data which describes background noise is acquired by at least one of at least one of the acceleration sensors or an additional sensor (also called auxiliary sensor), for example a sound pressure level sensor or microphone. The setup quality data is then determined based on the background noise data. For example, noise comparison data is acquired which describes a predetermined quantity of the noise contained in at least one of the signal noise data or the background noise data. Noise quality data is then determined based on the at least one of the signal noise data or the noise comparison data by comparing the noise to the predetermined (for example, acceptable) quantity of the noise, wherein the noise quality data is determined to indicate a predetermined (for example desired, specifically sufficient or good, or undesired, for example insufficient or bad) quality of the noise if the comparison results in that the noise has a predetermined relationship to (for example, is less than or equal to) the predetermined quantity of the noise.

For example, the setup quality data is determined to describe the predetermined quality of the positioning of the array of pressure sensors on the anatomical body part if the pressure quality data has been determined to indicate the predetermined quality of the pressure values detected by each of the pressure sensors and the pressure change quality data has been determined to indicate the predetermined quality of the first-order temporal derivative of the pressure values and the noise quality data has been determined to indicate the predetermined quality of the noise component.

For example, pressure change quality data is determined based on the pressure change data and the pressure change threshold data by comparing the first-order temporal derivative of the pressure values detected by each of the pressure sensors to the at least one threshold value of the first-order temporal derivative of the pressure values, wherein the pressure change quality data is determined to indicate a predetermined (for example desired, specifically sufficient or good) quality of the first-order temporal derivative of the pressure values if the comparison results in that the first-order temporal derivative of the pressure values has a predetermined relationship to the at least one threshold value of the first-order temporal derivative of the pressure values. For example, it is determined whether the pressure rise indicated by the first-order temporal derivative is constant, for example if the first-order temporal derivative is at least substantially zero, for example zero+/−a small (for example, negligible) value.

In a (for example third) exemplary step, signal noise data is determined based on the acceleration measurement data, wherein the signal noise data describes a noise component contained in the acceleration measurement data. For example, sensor noise is separated from the acceleration signal from which the acceleration measurement data is acquired by decomposing the acceleration signal.

In a (for example fourth) exemplary step, movement indication data is determined based on the acceleration measurement data, wherein the movement indication data describes whether the acceleration values indicate a gross movement of the patient's body. For example, movement acceleration threshold data is acquired which describes at least one threshold of the acceleration values indicating a gross movement of the patient's body, and the movement indication data is determined based on the acceleration measurement data and the movement acceleration threshold data. For example, the movement indication data is determined by comparing the acceleration values to the at least one threshold of the acceleration values indicating a gross movement of the patient's body, and determining that the movement indication data describes that an acceleration value indicates a gross movement of the patient's body if the acceleration value has a predetermined relationship to the at least one threshold of the acceleration values indicating a gross movement of the patient's body. For example, the movement indication data is determined additionally or alternatively by analysing the acceleration measurement data for low-frequency components in a time series of the of the acceleration values or acceleration values lying above a predetermined threshold which are considered to indicate such gross movement. For example, a variance of the acceleration values within segments of the acceleration measurement data of predetermined length is determined, and a variance exceeding a predetermined threshold is considered to indicate that the associated acceleration values have been caused at least partly by gross body motion. In an example, the movement indication data is determined from acceleration data acquired using an acceleration sensor which is not placed on the anatomical body part but elsewhere on the patient's body.

In a (for example fifth) exemplary step, setup quality data is determined based on the sensor contact data and the signal noise data and the movement indication data, wherein the setup quality data describes a for example predetermined (for example desired, specifically sufficient or good, or undesired, for example insufficient or bad) quality of the setup of the array of acceleration sensors on the anatomical body part. The setup quality data is for example related to a time signal.

In an example of the method according to the first aspect, heartbeat signal data is acquired which describes a time series of the heartbeat of the patient, wherein the setup quality data is determined based on the heartbeat signal data. The heartbeat of the patient is acquired for example using a heartbeat detector such as a photoplethysmograph or electrocardiograph on the patient.

This example comprises the following further optional steps:
a) determining, based on the heartbeat signal data, waveform correlation data describing a correlation of the waveform of the time series of the heartbeat;
b) acquiring correlation threshold data which describes at least one threshold value of the correlation of the waveform of the time series of the heartbeat;
c) determining, based on the heartbeat signal data, heartbeat spectrum data describing an energy spectrum of the time series of the heartbeat;
d) acquiring spectrum comparison data describing a predetermined energy spectrum of the time series of the heartbeat;
e) wherein the setup quality data describes the quality of the positioning of the heartbeat detector on the anatomical body part and is determined based on the waveform correlation data and the correlation threshold data and the heartbeat spectrum data and the spectrum comparison data.

Optionally, this example comprises the following further steps:
determining waveform quality data based on the waveform correlation data and the correlation threshold data by
comparing the correlation of the waveform of the time series of the heartbeat to the at least one threshold value of the correlation of the waveform of the time series of the heartbeat, wherein the waveform quality data is determined to indicate a predetermined quality of the waveform of the time series of the heartbeat if the comparison results in that waveform of the time series of the heartbeat has a predetermined relationship to the at least one threshold value of the correlation of the waveform of the time series of the heartbeat, and
determining the setup quality data based on the waveform quality data.

Optionally, this example comprises the following further steps:
determining heartbeat spectrum quality data based on the heartbeat spectrum data and the spectrum comparison data by
comparing the energy spectrum of the time series of the heartbeat to the predetermined energy spectrum of the time series of the heartbeat, wherein the heartbeat spectrum quality data is determined to indicate a predetermined quality of the first energy spectrum of the time series of the heartbeat if the comparison results in that the energy spectrum of the time series of the heartbeat has a predetermined relationship to the predetermined energy spectrum of the time series of the heartbeat, and
determining the setup quality data based on the heartbeat spectrum quality data.

In an example of the method according to the first aspect, the setup quality data is determined to describe a predetermined (for example desired, specifically sufficient or good, or undesired, for example insufficient or bad) quality of the positioning of the heartbeat detector on the anatomical body part if the waveform quality data has been determined to indicate the predetermined quality of the waveform of the time series of the heartbeat and the heartbeat spectrum quality data has been determined to indicate the predetermined (for example desired, specifically sufficient or good, or undesired, for example insufficient or bad) quality of the first energy spectrum of the time series of the heartbeat.

In an example, the method according to the first aspect comprises determining time-correlated measurement data describing a time-correlation of the acceleration measurement data with the setup quality data, and determining whether the data set comprising the time-correlated measurement data has a predetermined length of acceleration values which are associated with points in time at which the correlated setup quality data describes the predetermined quality of the setup of the array of acceleration sensors. The length of the data set indicates whether enough measurement values indicating the predetermined quality have been acquired.

In a second aspect, the invention is directed to a computer-implemented medical method of determining the validity of acceleration values sampled using an array of acceleration sensors placed on an anatomical body part of a patient. The method according to the second aspect comprises for example executing, on at least one processor of at least one computer (for example at least one computer being part of a portable system, for example a handheld device such as a mobile phone or tablet computer), the following exemplary steps which are executed by the at least one processor.

In a (for example, first) exemplary step, the method according to the first aspect is executed.

In a (for example, second) exemplary step, time-correlated measurement data is determined which describes a time-correlation of the acceleration measurement data with the setup quality data and, if the setup quality data associated with a specific point in time does not describe the predetermined quality of the setup of the array of acceleration sensors, saving an acceleration value associated with the specific point in time and marking the associated acceleration value that it shall not be used further, otherwise saving the acceleration value associated with the specific point in time. This allows using acceleration measurement data acquired during a pre-check for assessing the quality of the positioning of the array of acceleration sensors within the framework of later continuous processing of the setup quality data.

In a third aspect, the invention is directed to a computer program comprising instructions which, when the program is executed by at least one computer, causes the at least one computer to carry out method according to the first or second aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first or second aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the third aspect therefore may alternatively or additionally relate to a data stream representative of the aforementioned program, i.e. comprising the program.

In a fourth aspect, the invention is directed to a computer-readable storage medium on which the program according to the third aspect is stored. The program storage medium is for example non-transitory.

In a fifth aspect, the invention is directed to at least one computer (for example, a computer), comprising at least one processor (for example, a processor), wherein the program according to the third aspect is executed by the processor, or wherein the at least one computer comprises the computer-readable storage medium according to the fourth aspect. Alternatively or additionally, the invention according to the fifth aspect is directed to a for example non-transitory computer-readable program storage medium storing a program for causing the computer according to the fifth aspect to execute the data processing steps of the method according to the first or second aspect.

In a sixth aspect, the invention is directed to a medical system (for example, a system for cranial accelerometry), comprising:
a) the at least one computer according to the fifth aspect;
b) at least one electronic data storage device storing the acceleration data and the movement indication data, and for example the pressure threshold data and the pressure change threshold data, and for example the noise comparison data and for example the correlation threshold data and the spectrum comparison data; and
c) an acceleration sensor for receiving acceleration signals from an anatomical body part;
d) for example, a heartbeat detector for receiving the patient's heartbeat signals; and
e) for example, a pressure sensor for receiving the pressure values,
wherein the at least one computer is operably coupled to
   the at least one electronic data storage device for acquiring, from the at least one data storage device, for example the pressure threshold data and the pressure change threshold data, and for example the noise comparison data and for example the correlation threshold data and the spectrum comparison data,
   for example the pressure detector for receiving, from the pressure detector, pressure signals corresponding to the pressure values and generating, from the pressure signals, the contact pressure data, and
   the heartbeat detector, for receiving, from the heartbeat detector, the patient's heartbeat signals and generating, from the heartbeat signals, the heartbeat signal data.

In a seventh aspect, the invention is directed to use of the system according to the sixth aspect for conducting a medical procedure, wherein the use comprises execution of the steps of the method according to any one of the preceding method claims for determining the patient's physiological status.

For example, the invention does not involve or in particular comprise or encompass an invasive step which would represent a substantial physical interference with the body requiring professional medical expertise to be carried out and entailing a substantial health risk even when carried out with the required professional care and expertise.

### Definitions

In this section, definitions for specific terminology used in this disclosure are offered which also form part of the present disclosure.

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (VWWV) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. The monitor may also be the monitor of a portable, for example handheld, device such as a smart phone or personal digital assistant or digital media player or tablet computer.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy.

In order to distinguish the different data used by the present method, the data are denoted (i.e. referred to) as “XY data” and the like and are defined in terms of the information which they describe, which is then preferably referred to as “XY information” and the like.

### BRIEF DESCRIPTION OF THE DRAWINGS

In the following, the invention is described with reference to the appended figures which give background explanations and represent specific embodiments of the invention.

The scope of the invention is however not limited to the specific features disclosed in the context of the figures, wherein

FIG. 1 illustrates the basic steps of the method according to the first aspect;

FIG. 2 shows an embodiment of the present invention, specifically the method according to the first aspect;

FIG. 3 is a flow diagram illustrating a pre-check for checking the positioning of the array of acceleration sensors before initiating the actual acceleration measurement;

FIG. 4 is a flow diagram illustrating the process of conducting the acceleration measurement;

FIG. 5 gives an overview of the data processing flow in which the method according to the first aspect can be used;

FIG. 6 is a schematic illustration of the system according to the sixth aspect; and

FIG. 7 shows an embodiment of the system according to the sixth aspect in which a headset is attached to a patient's head.

### DESCRIPTION OF EMBODIMENTS

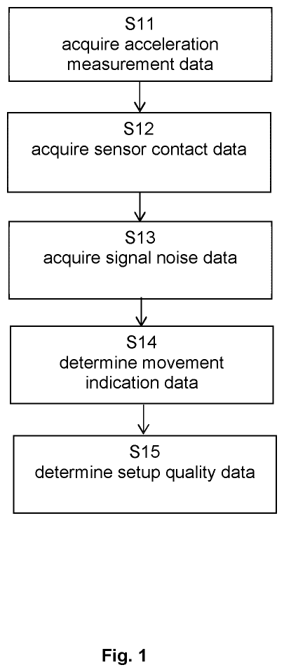

FIG. 1 illustrates the basic steps of the method according to the first aspect, in which step S11 encompasses acquisition of the acceleration measurement data, step S12 encompasses acquisition of the sensor contact data and subsequent step S13 encompasses acquisition of the signal noise data. The movement indication data is determined in step S14, and the setup quality data in subsequent step S15.

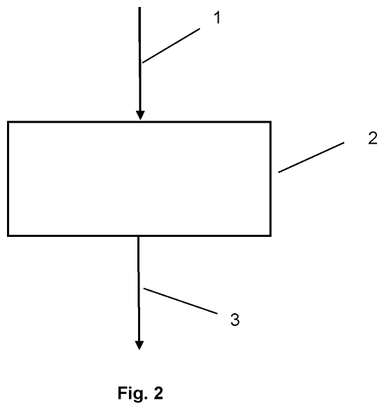

FIG. 2 illustrates an embodiment of the present invention that includes all essential features of the invention. In this embodiment, the entire data processing which is part of the method according to the first aspect is performed by a computer 2. Reference sign 1 denotes the input of data acquired by the method according to the first aspect into the computer 2 and reference sign 3 denotes the output of data determined by the method according to the first aspect.

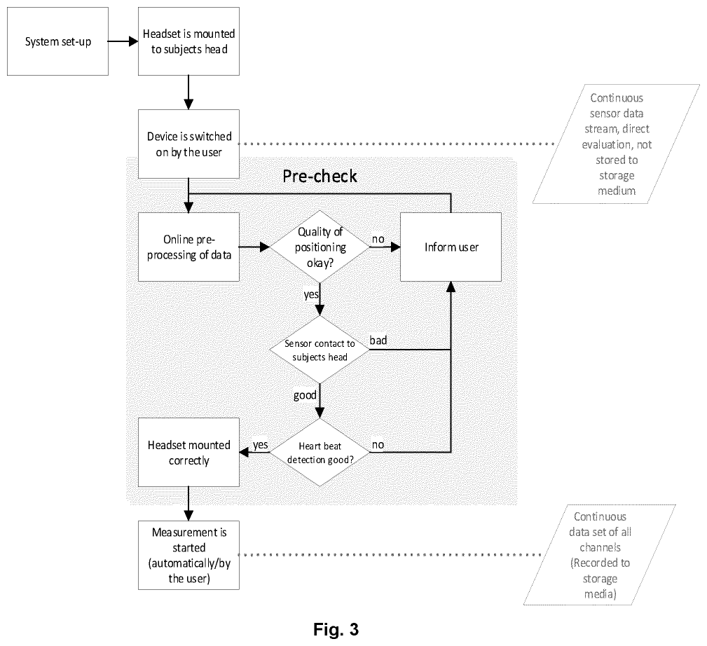

FIG. 3 illustrates the flow of setting up and pre-checking the functionality of a headset with acceleration sensors on a patient. After an initial system set-up, the headset is mounted to a subject's (i.e. patient's) head and switched on by a user. The device then determines whether a continuous data stream is present, which is evaluated directly without storing the data to a storage medium. A pre-check of the functionality of the system then starts which involves online pre-processing of acceleration measurement data and/or of data indicating the results of photoplethysmography conducted on the patient and/or measuring sound pressure and/or pressure values. This includes determining whether the quality of the positioning of the headset on the patient is acceptable. If this is not the case, the user is informed and given the opportunity to review and improve the positioning. If it is determined that the quality of the positioning is acceptable, the flow continues with determining whether the sensor contact to the subject's head is acceptable. If this is not the case, the user is informed and given the opportunity to review and improve the sensor contact. If it is determined that the quality of the sensor contact is acceptable, the flow continues with determining whether the heartbeat detection is acceptable. If this is not the case, the user is informed and given the opportunity to review and improve the arrangement of the heartbeat sensor. If it is determined that the quality of the heartbeat detection is acceptable, the flow continues with determining that the headset is mounted correctly on the patient. The actual taking of acceleration measurement values and/or of the data indicating the results of photoplethysmography conducted on the patient and/or measuring sound pressure and/or pressure values is than started automatically or by user interaction, and continuous data sets of all sensor channels are then recorded to a storage medium.

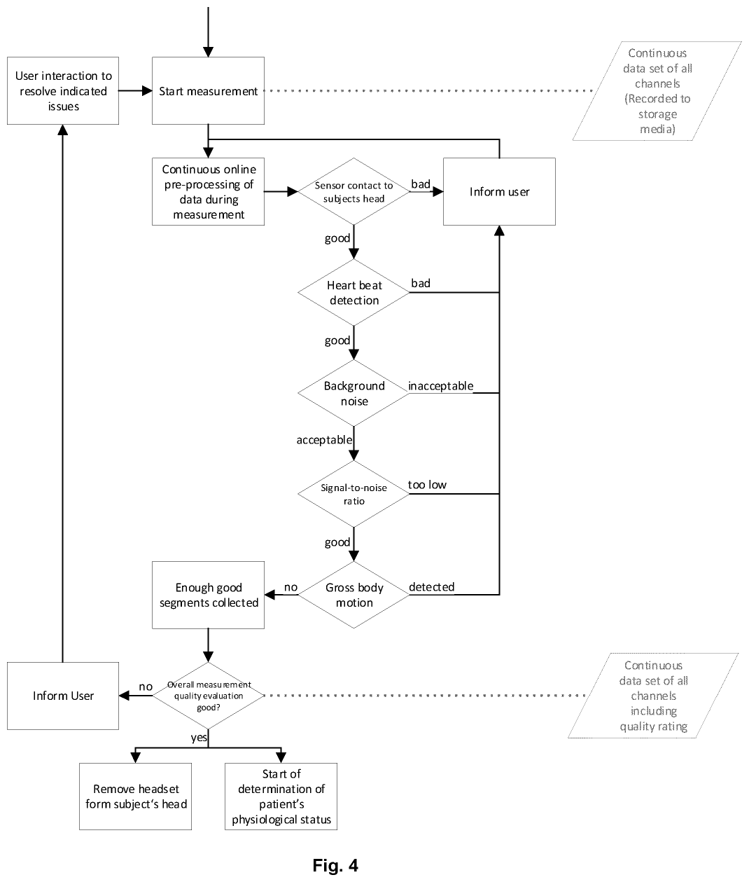

FIG. 4 illustrates the steps which occur subsequently to starting taking of the acceleration measurement values as well as of the data indicating the results of photoplethysmography conducted on the patient and measuring sound pressure and pressure values. A continuous online pre-processing of the data taken during the measurement (and for example acquired by the steps of the method according to the first aspect) is conducted before the data is stored and evaluated. The online pre-processing involves determining whether the sensor contact to the patient's head is (still, i.e. remains) to be good. If this is not the case and the contact is determined to be bad, the user is informed and given the opportunity to improve the contact. If it is determined that the contact is good, the method continues with determining whether the heartbeat detection is good. If this is not the case, the user is informed and given the opportunity to review and improve the arrangement of the heartbeat sensor. If it is determined that the heartbeat detection is good, the method continues with determining whether the background noise is acceptable. If this is not the case, the user is informed and given the opportunity to improve the background noise, for example by asking people around him for acoustic silence. If it is determined that the background noise is acceptable, it is determined whether the signal-to-noise ratio is good. If it is determined that this is not the case, i.e. that the signal-to-noise-ratio is too low, the user is informed and given the opportunity to remedy this situation, e.g. by re-positioning the headset on the patient. If it is determined that the signal-to-noise ratio is good, the method continues with determining whether a gross body motion of the patient has been detected. If it has been detected, the user informed and given the opportunity to ask the patient to keep still. If no gross body motion has been detected, the method continues with determining whether enough good data segments (i.e. segments of acceleration measurement data) have been collected to conduct a meaningful evaluation of the acceleration measurement data, and whether the overall quality of the conducted measurement is good, which is derived from a continuous data set of all measurement channels including a quality rating. If this is not the case, the user is informed and given the opportunity to interact and resolve any indicated issue impairing the measurement quality. If it is determined that the overall measurement quality is good, the headset is removed from the patient's head, and the acceleration measurement data is evaluated to determine the patient's physiological status, e.g. to determine whether the patient has suffered a stroke, particularly the type of stroke which he may suffered such as a hemorrhagic stroke. The data processing steps described in the context of FIG. 4 may also conducted in any different order, with the conditions for their execution being appropriately adapted.

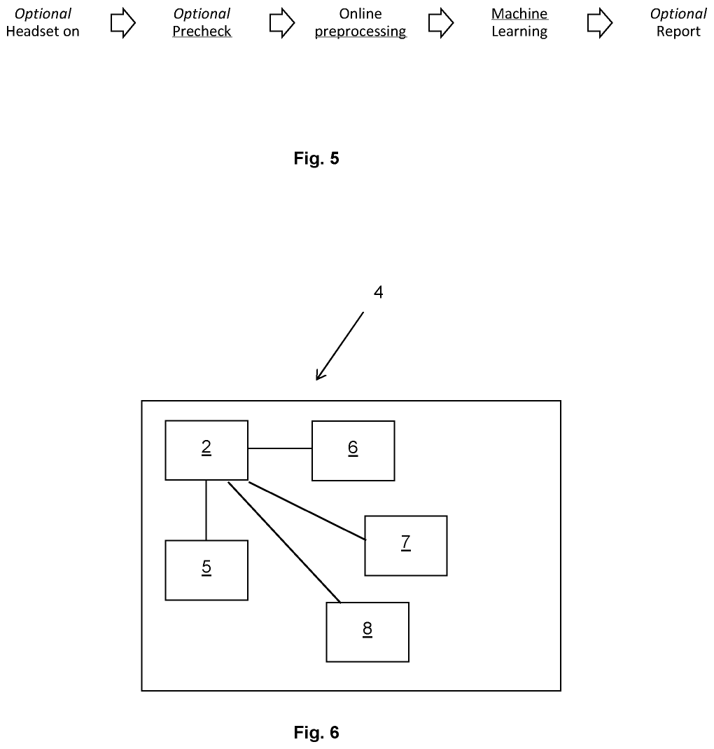

FIG. 5 illustrates a complete workflow which lies within the scope of the disclosed invention and includes steps which are performed subsequently to the above-describes data processing. After placing the headset on the patient and conducting the above-explained pre-check and online pre-processing: after conducting for example artefact removal from the acceleration measurement data, the measurement signals are segmented into a part indicating a heartbeat signal and a part indicating another acceleration-induced signal component, which is followed by signal decomposition according to frequency and optionally feature augmentation. Subsequently, an accordingly configured machine learning classifier is used to determine, on the basis of the result of the foregoing data processing, the patient's physiological status. Optionally, a report about the results and for example the metainformation characterising the measurement is then generated.

FIG. 6 is a schematic illustration of the medical system 4 according to the sixth aspect. The system is in its entirety identified by reference sign 4 and comprises a computer 2, an electronic data storage device (such as a hard disc) 5 for storing at least the patient data and an acceleration sensor 6, and optionally a heartbeat detector 7 and a pressure sensor 8. The components of the medical system 4 have the functionalities and properties explained above with regard to the sixth aspect of this disclosure.

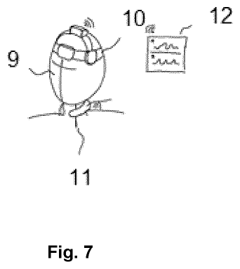

FIG. 7 shows an embodiment of the system according to the sixth aspect which comprises a headset 10 with acceleration sensor which is attached to the patient's head 9. Optionally, the embodiment comprises a heartbeat detector 11 which is embodied by for example a neck collar and suitable to measure e.g. the carotid pulse. The headset is for example configured for wireless transmission of data derived from measurement signals to an information processing and display device 12, which is for example handheld.

An additional explanation of the system lying in the scope of the present invention is presented in the following.

The system comprises various subsystem components that include:
a headset that is patient-contacting and includes:
   one or more photoplethysmography (PPG) sensors for detecting the heartbeat, heart rate, and timing;
   one or more sound pressure level (SPL) sensors for detecting ambient environment noise; and
   a plurality of accelerometer sensors to detect the acceleration at dedicated locations around the patient's head.
a data collector which digitizes the analogue sensor signals (either as an individual component or integrated into the sensor elements);
a computer unit which incorporates the device software and space to store the recording data and;
device software, which provides the user interface, hardware control, software libraries, and algorithms for signal preparation, signal processing, signal separation and classification for a plurality of clinical indications

The system collects and stores sensor data caused by, for example the pulsatile blood flow from the cardiac cycle, leading to a slight acceleration of the skull. The system uses for example piezoelectric-based accelerometer sensors that measure a variety of signal components originating from the response of the head/brain to the blood flow. The plurality of acceleration sensors sense the motion and the data collector digitizes the signal. The computer unit provides the user interface, stores the data, performs the signal separation and the classification of the recorded data specific to the clinical indication.

A user places the headset of the device on a patient and sets up the user interface to perform a recording. Typically, the user will perform a recording that is approximately one to two minutes long, though in some cases, if the patient moves or displaces the headset, the recording may be prolonged. The device software analyzes the recording and separates the recorded signal into its signal components. Predictive features are calculated for each signal components and these are then classified by statistical and/or machine learning approaches using known thresholds, data of known conditions, etc. The device software then displays the result of classification into defined clinical indications to the user.

It is preferred that the recording is mostly free of artefacts which could interfere with for example classification. Disclosed are methods for quality analysis of the signal before the recording starts and continuous quality check during a recording.
The headset is mounted on a subject's head and the device is switched on. Before the recording is started, a pre-check is performed using a temporary data stream of the system. The pre-check consists out of several steps including online (i.e., during the actual measurement) pre-processing to determine:
The quality of the headset positioning.
   The plurality of sensors is checked by a position measurement system to ensure proper alignment and angles of the sensors relative to each other using specified ranges.
Sensor functionality after the headset was placed on the subject's head: to test proper functioning (for example power supply) of the plurality of all sensors, the energy the sensors are determined and checked against a threshold.
   If a sensor falls below a threshold, the user is informed. The process is repeated until all sensors function properly.
Sensor contact to subject's head: The quality of the signal depends on proper contact to the subject's head.
   The plurality of sensors is checked for proper contact to the subject's head. Proper contact is checked for example by pressure sensors. Using the aforementioned pressure sensors, the proper contact is measured by pressing the contact surface of a sensor onto the subject's head, such that the pressure in the closed system rises. This is measured by a pressure sensor. A constant pressure rise indicates the contact force. If the quality for a given sensor falls below a threshold the contact is deemed insufficient. In this case, the user is informed such that the system containing the plurality of sensors can be rearranged until a sufficient contact is achieved.
   Acquired signals can be decomposed using decomposition methods (e. g. principal component analysis, independent component analysis, blind source separation, spectral subtraction, adaptive noise cancelling, noise modelling via convolutional neural networks etc.). Decomposed signals can be used to quantify the quality of individual sensors and the overall recording.
   The PPG sensor or plurality of PPG sensors for heartbeat detection is checked for proper function by determining at least one of the following cumulatively or alternatively:
      waveform correlations (e.g. Fisher correlation, Pearson correlation)
      energy thresholds (dead sensors fall below a lower boundary threshold, improper attachment surpasses an upper threshold due to ambient stray light on the PPG sensor)
      energy within characteristic frequency ranges is determined as noise
      energy within other characteristic frequency ranges is determined as signal
      In some cases heartbeats can be detected directly via an acceleration sensor.
      If correlation, energy thresholds, signal-to-noise ratio are not of sufficient quality, the user is informed to readjust the sensor or plurality of sensors for heartbeat monitoring.
If the pre-check is successful, the user is informed that the system is mounted correctly to the subject's head, that the sensors signals are of sufficient quality, and that the recording can be started.
After start of the recording, the pre-checks are continuously performed throughout the recording.
If pre-check parameters change and thus data quality changes to being insufficient, the affected segment is marked accordingly and the user is informed. The user is informed when the pre-check parameters are of sufficient quality.
In addition to pre-check parameters, further data quality parameters are checked during the recording, which include:
   Gross body motion: The peak-to-peak amplitude and the segment variance are used to determine if segments are outside of acceptable threshold. Furthermore, the signal can be compared against an event library containing known motion artifacts (blinking, swallowing, sneezing, coughing etc.). Using statistical or machine learning algorithms this library can be compared against the recorded signal.
      If gross body motion is detected the user is informed with live feedback from the clinical software. The heartbeats affected by gross body motion are marked in the clinical software and will be excluded from the analysis.
   Background noise and sounds: Background noise and sounds (speech, beeping of vital monitors, breathing of the subject etc.) are detected by determining a measure of similarity between the SPL sensor and the plurality of acceleration sensors (e. g. coherence analysis). Coherence with the SPL channel is determined for each acceleration channel individually. Furthermore, signals can be decomposed using decomposition methods (e. g. principal component analysis, independent component analysis, blind source separation, spectral subtraction, adaptative noise cancelling, noise modelling via convolutional neural networks etc.)
      If a threshold is surpassed or background noise is identified via decomposition, the recording quality is deemed as insufficient, and the user is informed to enforce countermeasures (e. g. ensuring that talking during recording is reduced or machine sound such as beeping is turned off). The heartbeats affected by background noise are marked in the clinical software. They will be either excluded from the analysis or the respective sound frequencies are removed and the original waveform is restored. In the latter case, the segment will be used for analysis.
   Signal-to-noise ratio for plurality of acceleration sensors: The noise and the signal are modelled using neural networks.
      If the signal-to-noise ratio falls below a threshold, the affected segment is marked accordingly and the user is informed to readjust the headset.
   If a sufficient number segments of high quality are collected, the overall recording quality is deemed as good and the user is informed that the recording can be stopped or the recording is stopped automatically. If an insufficient number of high-quality segments were collected after a given time, the user is informed and the recording can be repeated.
When enough segments of high quality were collected, the user will remove the headset from the subject's head.
Devices contained in the system are for example:
   a headset containing:
      one or more PPG sensors
      one or more SPL sensors
      a plurality of acceleration sensors
   a data collector
   a computer unit; and
   device software.

1. A computer-implemented medical method of analyzing the setup of an array of acceleration sensors on an anatomical body part of a patient, the method comprising:
acquiring by acceleration sensors acceleration measurement data that describes acceleration values;
acquiring sensor contact data that describes a quality of a contact between at least one of the acceleration sensors comprised in the array of acceleration sensors and the anatomical body part of the patient;
determining signal noise data based on the acceleration measurement data, wherein the signal noise data describes a noise component contained in the acceleration measurement data;
determining movement indication data based on the acceleration measurement data, wherein the movement indication data describes whether the acceleration values indicate a gross movement of the patient's body; and
determining setup quality data based on the sensor contact data the signal noise data, and the movement indication data, wherein the setup quality data describes a quality of the setup of the array of acceleration sensors on the anatomical body part.

2. The method according to claim 1, wherein the determining the sensor contact data comprises:
acquiring contact pressure data that describes pressure values detected by each of pressure sensors comprised in an array of pressure sensors after positioning each of the pressure sensors on the anatomical body part;
acquiring pressure threshold data that describes at least one threshold value of the pressure values detected by each of the pressure sensors;
determining pressure change data based on the contact pressure data, wherein the pressure change data describes a first-order temporal derivative of the pressure values detected by each of the pressure sensors; and
acquiring pressure change threshold data that describes at least one threshold value of the first-order temporal derivative of the contact pressure data.

3. The method according to claim 2, wherein the array of pressure sensors and the array of acceleration sensors are comprised in the same device and for example have a predetermined, for example at least one of fixed or known, spatial relationship to each other.

4. The method according to claim 1, wherein the sensor contact data is acquired based on the acceleration measurement data, by one or more of:
acceleration sensor combinations, correlation of ambient sound on the acceleration measurement data or by principal component analysis or independent component analysis to determine signal and noise components on the acceleration measurement data, or by skin resistance measurement.

5. The method according to claim 2, further comprising:
determining pressure quality data based on the contact pressure data and the pressure threshold data by comparing the pressure values detected by each of the pressure sensors to the at least one threshold value of the pressure values,
wherein the pressure quality data is determined to indicate a predetermined quality of the pressure values detected by each of the pressure sensors if the comparison results in that the pressure values detected by each of the pressure sensors have a predetermined relationship to the at least one threshold value of the pressure values.

6. The method according to claim 2, further comprising:
determining pressure change quality data based on the pressure change data and the pressure change threshold data by comparing the first-order temporal derivative of the pressure values detected by each of the pressure sensors to the at least one threshold value of the first-order temporal derivative of the pressure values,
wherein the pressure change quality data is determined to indicate a predetermined quality of the first-order temporal derivative of the pressure values if the comparison results in that the first-order temporal derivative of the pressure values has a predetermined relationship to the at least one threshold value of the first-order temporal derivative of the pressure values.

7. The method according to claim 1, further comprising acquiring, using at least one of at least one of the acceleration sensors or an auxiliary sensor comprising a pressure level sensor, background noise data that describes background noise,
wherein the setup quality data is determined based on the background noise data.

8. The method according to claim 1, further comprising:
acquiring noise comparison data that describes a predetermined quantity of the noise contained in at least one of the signal noise data or the background noise data; and
determining noise quality data based on the at least one of the signal noise data or the noise comparison data by comparing the noise to the predetermined quantity of the noise,
wherein the noise quality data is determined to indicate a predetermined quality of the noise if the comparison results in that the noise has a predetermined relationship to the predetermined quantity of the noise.

9. The method according to claim 7 wherein the setup quality data is determined to describe a predetermined quality of the positioning of the array of pressure sensors on the anatomical body part if the pressure quality data has been determined to indicate the predetermined quality of the pressure values detected by each of the pressure sensors and the pressure change quality data has been determined to indicate the predetermined quality of the first-order temporal derivative of the pressure values and the noise quality data has been determined to indicate the predetermined quality of the noise component.

10. The method according to claim 1, further comprising acquiring heartbeat signal data that describes a time series of the heartbeat of the patient,
wherein the setup quality data is determined based on the heartbeat signal data.

11. The method according to claim 10, further comprising:
one or more of:
determining, based on the heartbeat signal data, waveform correlation data describing a correlation of the waveform of the time series of the heartbeat;
acquiring correlation threshold data which describes at least one threshold value of the correlation of the waveform of the time series of the heartbeat;
determining, based on the heartbeat signal data, heartbeat spectrum data describing an energy spectrum of the time series of the heartbeat; and/or
acquiring spectrum comparison data describing a predetermined energy spectrum of the time series of the heartbeat,
wherein the setup quality data describes the quality of the positioning of a heartbeat detector on the anatomical body part and is determined based on the at least one of the waveform correlation data or the correlation threshold data and the heartbeat spectrum data or the spectrum comparison data.

12. The method according to claim 11, further comprising:
determining waveform quality data based on the waveform correlation data and the correlation threshold data by comparing the correlation of the waveform of the time series of the heartbeat to the at least one threshold value of the correlation of the waveform of the time series of the heartbeat,
wherein the waveform quality data is determined to indicate a predetermined quality of the waveform of the time series of the heartbeat if the comparison results in that waveform of the time series of the heartbeat has a predetermined relationship to the at least one threshold value of the correlation of the waveform of the time series of the heartbeat, and wherein the setup quality data is determined based on the waveform quality data.

13. The method according to claim 11, further comprising:
determining heartbeat spectrum quality data based on the heartbeat spectrum data and the spectrum comparison data by comparing the energy spectrum of the time series of the heartbeat to the predetermined energy spectrum of the time series of the heartbeat,
wherein the heartbeat spectrum quality data is determined to indicate a predetermined quality of the first energy spectrum of the time series of the heartbeat if the comparison results in that the energy spectrum of the time series of the heartbeat has a predetermined relationship to the predetermined energy spectrum of the time series of the heartbeat,
wherein the setup quality data is determined based on the heartbeat spectrum quality data.

14. The method according to claim 10, further comprising determining the setup quality data to describe a predetermined quality of the positioning, on the anatomical body part, of a heartbeat detector used to acquire the heartbeat of the patient if the waveform quality data has been determined to indicate the predetermined quality of the waveform of the time series of the heartbeat and the heartbeat spectrum quality data has been determined to indicate the predetermined quality of the first energy spectrum of the time series of the heartbeat.

15. A computer-implemented medical method of determining the validity of acceleration values sampled using an array of acceleration sensors placed on an anatomical body part of a patient, the method comprising:
acquiring by acceleration sensors acceleration measurement data that describes acceleration values;
acquiring sensor contact data that describes a quality of a contact between at least one of the acceleration sensors comprised in the array of acceleration sensors and the anatomical body part of the patient;
determining signal noise data based on the acceleration measurement data, wherein the signal noise data describes a noise component contained in the acceleration measurement data;
determining movement indication data based on the acceleration measurement data, wherein the movement indication data describes whether the acceleration values indicate a gross movement of the patient's body;
determining setup quality data based on the sensor contact data, the signal noise data, and the movement indication data, wherein the setup quality data describes a quality of the setup of the array of acceleration sensors on the anatomical body part; and
determining time-correlated measurement data describing a time-correlation of the acceleration measurement data with the setup quality data and, if the setup quality data associated with a specific point in time does not describe the predetermined quality of the setup of the array of acceleration sensors, saving an acceleration value associated with the specific point in time and marking the associated acceleration value that it shall not be used further, otherwise saving the acceleration value associated with the specific point in time.

16. The method according to claim 15, further comprising:
acquiring movement acceleration threshold data describing at least one threshold of the acceleration values indicating a gross movement of the patient's body; and
determining the movement indication data based on the acceleration measurement data and the movement acceleration threshold data.

17. The method according to claim 15 further comprising, determining the movement indication data by comparing the acceleration values to the at least one threshold of the acceleration values indicating a gross movement of the patient's body, and determining that the movement indication data describes that an acceleration value indicates a gross movement of the patient's body if the acceleration value has a predetermined relationship to the at least one threshold of the acceleration values indicating a gross movement of the patient's body.

18. The method according to claim 15, further comprising
determining time-correlated measurement data describing a time-correlation of the acceleration measurement data with the setup quality data, and determining whether the data set comprising the time-correlated measurement data has a predetermined length of acceleration values which are associated with points in time at which the correlated setup quality data describes the predetermined quality of the setup of the array of acceleration sensors.

19. (canceled)

20. A non-transient computer-readable storage medium on which a program is stored and that is executable by a processor to carry out a method comprising:
acquiring by acceleration sensors acceleration measurement data that describes acceleration values;
acquiring sensor contact data that describes a quality of a contact between at least one of the acceleration sensors comprised in the array of acceleration sensors and the anatomical body part of the patient;
determining signal noise data based on the acceleration measurement data, wherein the signal noise data describes a noise component contained in the acceleration measurement data;
determining movement indication data based on the acceleration measurement data, wherein the movement indication data describes whether the acceleration values indicate a gross movement of the patient's body; and
determining setup quality data based on the sensor contact data, the signal noise data, and the movement indication data, wherein the setup quality data describes a quality of the setup of the array of acceleration sensors on the anatomical body part.

21. A computer comprising:
at least one processor;
a non-transient memory device operatively coupled with the processor and storing a program thereon,
wherein the processor is configured to execute the program to analyze the setup of an array of acceleration sensors on an anatomical body part of a patient to:
acquire by acceleration sensors acceleration measurement data that describes acceleration values;
acquire sensor contact data that describes a quality of a contact between at least one of the acceleration sensors comprised in the array of acceleration sensors and the anatomical body part of the patient:
determine signal noise data based on the acceleration measurement data, wherein the signal noise data describes a noise component contained in the acceleration measurement data;
determine movement indication data based on the acceleration measurement data, wherein the movement indication data describes whether the acceleration values indicate a gross movement of the patient's body; and
determine setup quality data based on the sensor contact data, the signal noise data, and the movement indication data, wherein the setup quality data describes a quality of the setup of the array of acceleration sensors on the anatomical body part.

22. (canceled)

23. (canceled)

24. (canceled)

25. (canceled)

26. The method according to claim 1 further comprising:
generating a signal that indicates to an associated user whether the quality of the setup fulfills a predetermined criterion.
